(19) United States
(12) Patent Application Publication
(10) Pub. No.: US 2013/0123712 A1
(43) Pub. Date: May 16, 2013

(54) Plunger Rod Retaining Anchors

(71) Applicant: Becton, Dickinson and Company, Franklin Lakes, NJ (US)

(72) Inventor: Milan Ivosevic, Kinnelon, NJ (US)

(73) Assignee: Becton, Dickinson and Company, Franklin Lakes, NJ (US)

(21) Appl. No.: 13/669,724

(22) Filed: Nov. 6, 2012

Related U.S. Application Data
(60) Provisional application No. 61/558,581, filed on Nov. 11, 2011.

(51) Int. Cl. A61M 5/315

(52) U.S. Cl. 604/220; 604/218

(57) ABSTRACT

A plunger rod includes: an elongated body formed by a plurality of orthogonally spaced elongated ribs extending along a longitudinal axis; a first ledge adjacent a first end of the elongated body; a second ledge adjacent a second end of the elongated body; and an attachment portion extending from the second ledge. The second end of the elongated body opposes the first end of the elongated body. At least one elongated rib includes a retaining anchor positioned adjacent the second end adjacent the second ledge.

### BACKGROUND OF THE INVENTION

1. Field of the Invention

The invention relates, in general, to a plunger rod for use with a syringe and, more particularly, to a plunger rod that includes a plurality of anchors around the circumference thereof to prevent the plunger rod from being pulled out of the syringe barrel during aspiration of the syringe.

2. Description of Related Art

Current syringe designs can typically be categorized as two-piece syringes or three-piece syringes. A typical three-piece syringe includes a tubular barrel having an access opening formed at one end, and a smaller discharge opening formed at the opposing end. The lead end of an elongated plunger rod is received within the access opening of the barrel so as to be slidable within the barrel. Attached to the lead end of the plunger rod is a flexible sealing member or stopper that snugly seals against the interior surface of the barrel. A needle, a threaded member, or a non-threaded member is usually attached to the discharge opening on the barrel. The needle can be used to penetrate a surface while the threaded member can be used to attach the syringe to another medical device, such as a catheter. The flexible stopper is usually manufactured from an elastomeric material, such as rubber or a cross-linked or thermoplastic elastomer.

A two-piece syringe, on the other hand, includes a “stopper” that is in the form of a rigid sealing disc, also known as a plunger rod head. It is typically made of the same rigid plastic as the rest of the plunger rod. The sealing force in a two-piece syringe comes from a thin elastic barrel that deforms around the rigid plunger rod head.

During use, the discharge end of the syringe is initially placed in contact with a fluid. For example, the needle on the syringe can be inserted into a liquid medication. As the plunger is retracted within the barrel, a process known as aspiration, a negative pressure is formed within the end of the barrel so as to cause the fluid to be drawn into the barrel. The syringe can then be moved to a second location where advancing the plunger within the barrel causes the fluid to be pushed or expressed out of the discharge end of the barrel.

With reference to FIG. 1, a conventional plunger rod 1 includes an elongated body 3 formed by orthogonally spaced elongated ribs 5. A ledge 7 at one end provides a rest for a user's thumb. At the opposing end of the elongated body 3, an attachment portion 9 integrally extends from a ledge 11. The attachment portion 9 allows for a stopper (not shown) to be coupled thereto. The elongated body 3 also includes a retaining ring 13 positioned adjacent to the bottom surface of the ledge 11. The retaining ring 13 is provided to prevent the plunger rod 1 from being removed from the syringe barrel by interacting with a retaining structure provided at an end of the syringe barrel.

The current retaining ring provided on conventional plunger rods suffers from various deficiencies. For instance, a large amount of material is required to mold such plunger rods that include retaining rings. In addition, due to the complex geometry of such plunger rods, the molding of these items requires a large number of knockout pins to eject the molded plunger rod from the mold and a high molding cycle time.

Accordingly, a need exists for a plunger rod design that includes a feature having a simple geometry formed from a reduced amount of material for preventing the plunger rod from being removed from the syringe barrel.

### SUMMARY OF THE INVENTION

An object of the present invention is to provide a plunger rod having a feature for preventing the plunger rod from being removed from the syringe barrel. More specifically, a need exists for a plunger rod that includes a plurality of retaining anchors to replace the conventional retaining ring. The use of such retaining anchors reduces overall material use by 3%-5%, thereby yielding a “greener” syringe. In addition, using these retaining anchors instead of the conventional retaining ring leads to improved plunger rod moldability due to the less complex geometry and a reduction in the number of knockout pins required to eject the plunger rod from the mold which makes the mold more cost effective and robust.

Accordingly, provided is a plunger rod that includes: an elongated body formed by a plurality of orthogonally spaced elongated ribs extending along a longitudinal axis; a first ledge adjacent a first end of the elongated body; a second ledge adjacent a second end of the elongated body; and an attachment portion extending from the second ledge. The second end of the elongated body opposes the first end of the elongated body. At least one elongated rib includes a retaining anchor positioned adjacent the second end adjacent the second ledge.

According to one aspect of the present invention, each of the elongated ribs may include a retaining anchor positioned adjacent the second end adjacent the second ledge. The retaining anchors may be configured to prevent the elongated body from being removed from a syringe barrel by interacting with a retaining structure provided at an end of the syringe barrel.

According to another aspect of the invention, the retaining anchor may include a first angled portion extending from the second ledge toward the second end of the elongated body, a second portion extending parallel to one of the orthogonally spaced elongated ribs toward the second end of the elongated body, and a third portion extending perpendicularly to one of the orthogonally spaced elongated ribs. The first angled portion may extend from the second ledge at an angle of about 3° to about 60°. The third portion of the retaining anchor may interact with a retaining structure of a syringe barrel to prevent the plunger rod from being removed from the syringe barrel. Alternatively, the at least one retaining anchor may have a cross-sectional shape selected from the group consisting of: rectangular, rounded, or angled shape.

The plunger rod may be manufactured from a polymeric material selected from the group consisting of: polyolefines (such as polyethylene and polypropylene), polyamides, polyesters, polystyrene, polyurethane, polycarbonate, acrylonitrile-butadiene-styrene, fluoropolymers, ionomers, polyacrylates, or any combination thereof.

The attachment portion may be configured to secure a stopper thereto. The stopper and the plunger rod may be manufactured integrally or separately and from the same or different material.

In addition, provided is a syringe that includes: a substantially cylindrical syringe barrel comprising a fluid dispensing end and an open end. The open end includes a retaining structure. The syringe also includes a stopper configured to be received within the open end of the syringe barrel; and a plunger rod configured to engage a portion of the stopper. The plunger rod includes: an elongated body formed by a plurality of orthogonally spaced elongated ribs extending along a longitudinal axis; a first ledge adjacent a first end of the elongated body; a second ledge adjacent a second end of the elongated body; and an attachment portion extending from the second ledge. The second end of the elongated body opposes the first end of the elongated body. The attachment portion is configured to engage a portion of the stopper. At least one elongated rib includes a retaining anchor adjacent the second end adjacent the second ledge for engaging the retaining structure of the syringe barrel.

Further provided is a plunger rod and stopper assembly adapted for use with a syringe barrel. The assembly includes: a stopper; and a plunger rod configured to engage a portion of the stopper. The plunger rod includes: an elongated body formed by a plurality of orthogonally spaced elongated ribs extending along a longitudinal axis; a first ledge adjacent a first end of the elongated body; a second ledge adjacent a second end of the elongated body; and an attachment portion extending from the second ledge. The second end of the elongated body opposes the first end of the elongated body. The attachment portion is configured to engage a portion of the stopper. At least one elongated rib includes a retaining anchor adjacent the second end adjacent the second ledge.

These and other features and characteristics of the present invention, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and the claims, the singular form of “a”, “an”, and “the” include plural referents unless the context clearly dictates otherwise.

### BRIEF DESCRIPTION OF THE DRAWINGS

FIG. 1 is a perspective view of a conventional plunger rod;

FIG. 2 is a perspective view of a plunger rod having retaining anchors in accordance with the present invention;

FIG. 3 is an enlarged partial perspective view of the plunger rod having the retaining anchors of FIG. 2;

FIG. 4 is a side view of the plunger rod having the retaining anchors of FIG. 2;

FIG. 5 is a cross-sectional side view of the plunger rod of FIG. 2 attached to a stopper and positioned within a syringe barrel;

FIG. 6 is a cross-sectional side view of the plunger rod of FIG. 2 attached to a stopper and positioned within a syringe barrel with the plunger rod fully retracted; and

FIG. 7 is an enlarged partial cross-sectional view of the plunger rod of FIG. 6.

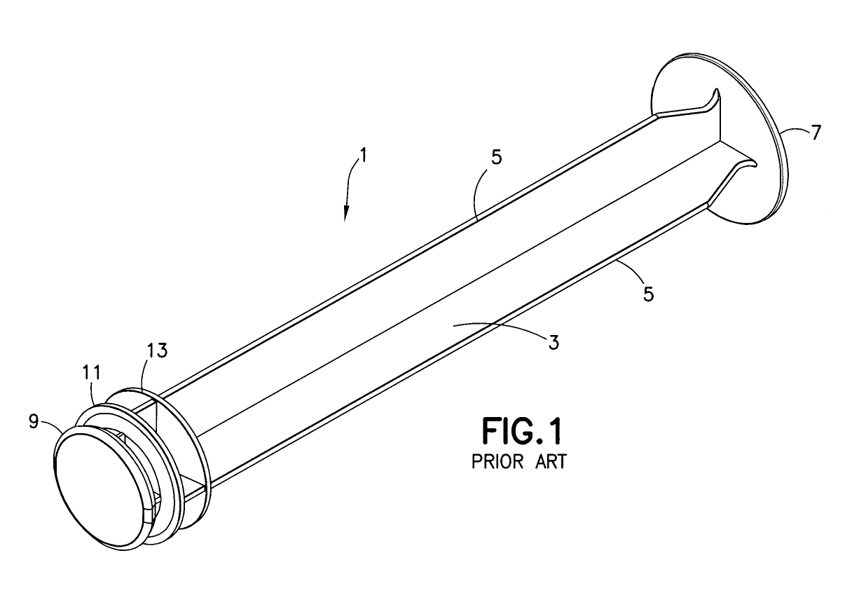

### DESCRIPTION OF THE INVENTION

For purposes of the description hereinafter, the terms “upper”, “lower”, “right”, “left”, “vertical”, “horizontal”, “top”, “bottom”, “lateral”, “longitudinal”, and derivatives thereof, shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

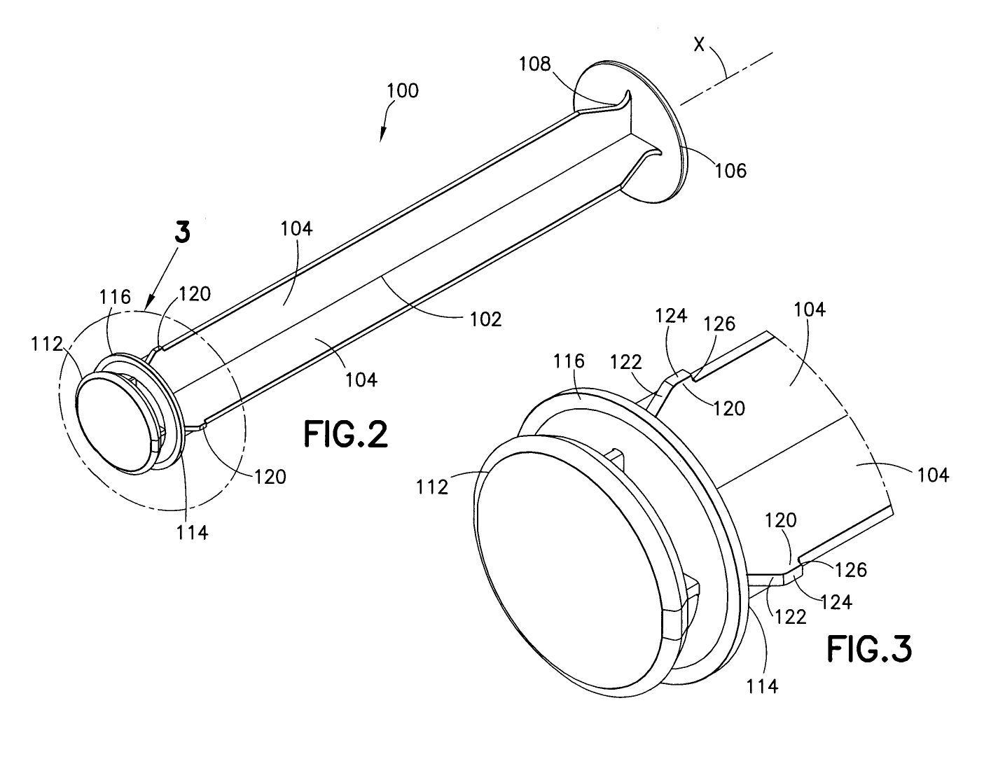

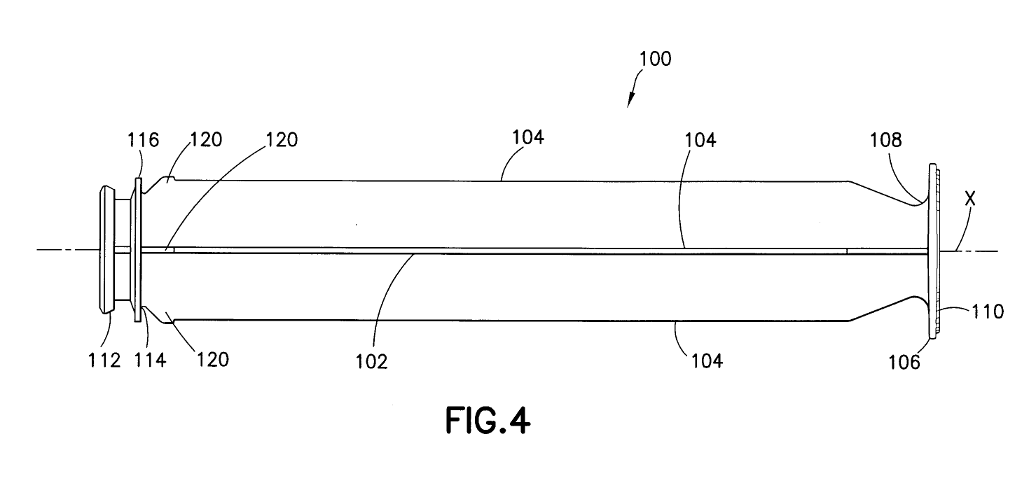

With reference to FIGS. 2-4, a plunger rod, generally denoted as reference numeral 100, includes an elongated body 102 formed by orthogonally spaced elongated ribs 104 extending along a longitudinal axis X. A first ledge 106 is provided at a first end 108 of the elongated body 102 for providing a rest for a user's thumb. A tactile surface 110 may be provided on a top surface of the first ledge 106 to increase the user's grip of the first ledge 106. An attachment portion 112 is positioned at a second, opposing end 114 of the elongated body 102 extending from a second ledge 116.

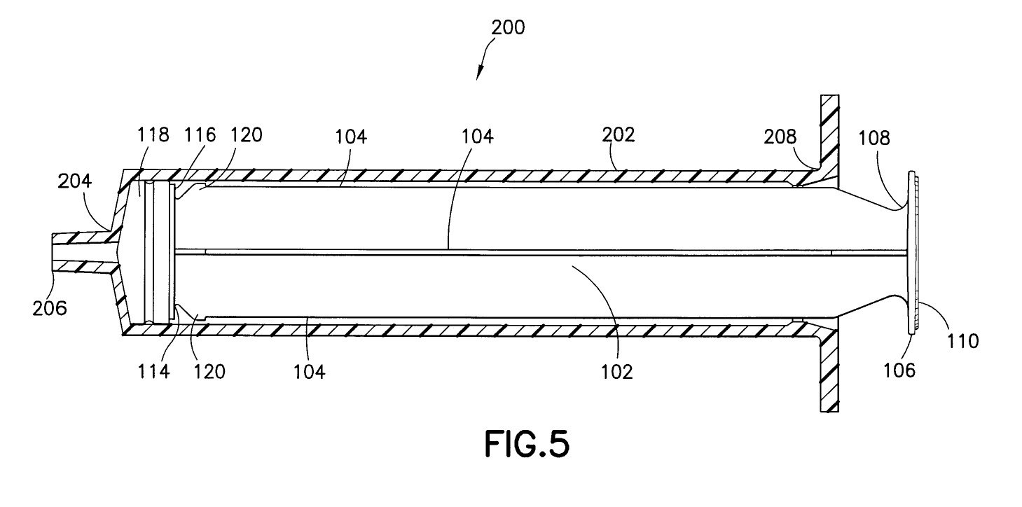

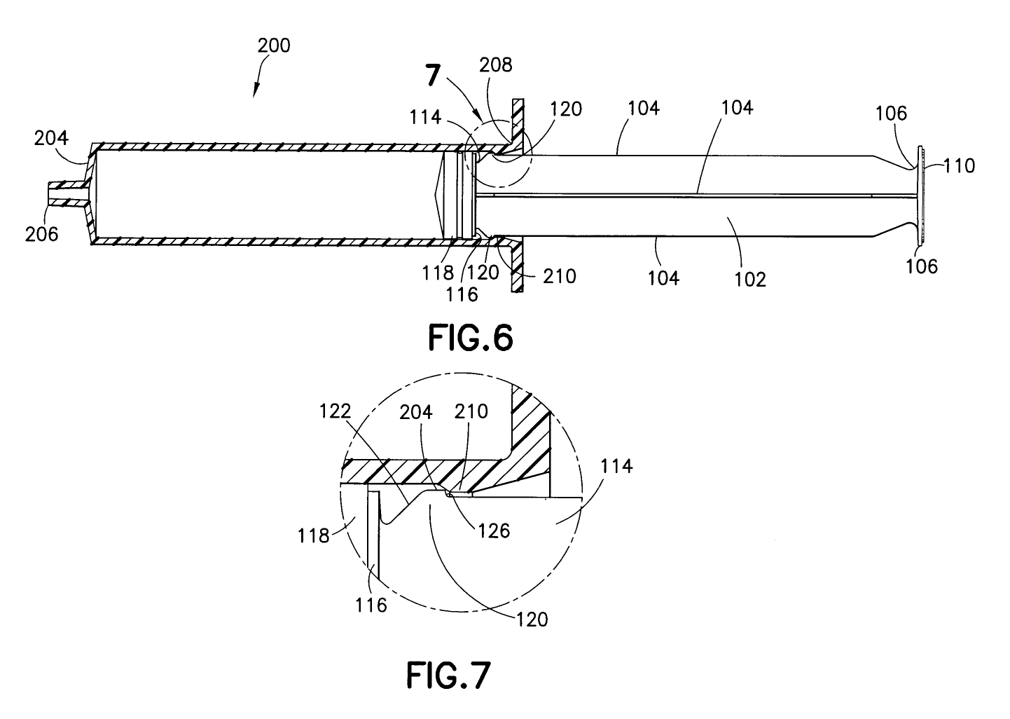

The attachment portion 112 is configured to receive an open bottom end of a stopper 118 (see FIGS. 5 and 6). The attachment portion 112 may have any suitable shape for securely coupling the stopper 118 thereto, such as shapes disclosed in co-pending United States Patent Publication Nos. 2012/0253291 and 2012/0253292, each filed on Mar. 28, 2012, which are hereby incorporated by reference in their entirety. In addition, the stopper 118 may have any suitable configuration such as the configurations disclosed in co-pending United States Patent Publication Nos. 2012/0253291 and 2012/0253292. Alternatively, the stopper 118 may be integrally molded with the plunger rod 100.

The plunger rod 100 also includes at least one retaining anchor 120 positioned adjacent to a bottom surface of the second ledge 116 on each of the orthogonally spaced ribs 104. Desirably, each of the ribs 104 includes one retaining anchor 120. According to one aspect of the present invention and with specific reference to FIG. 3, each of the retaining anchors 120 may include a first angled portion 122 extending from the second ledge 116 toward the second end 114 of the elongated body 102, a second portion 124 extending parallel to one of the orthogonally spaced elongated ribs 104 toward the second end 114 of the elongated body 102, and a third portion 126 extending perpendicularly to one of the orthogonally spaced elongated ribs 104. The first angled portion 122 may extend from the second ledge 116 at an angle of about 3° to about 60°. Alternatively, the retaining anchors 120 may have any other suitable cross-sectional shape such as, but not limited to, rectangular, rounded, or angled shape. The retaining anchors 120 are integrally molded with the plunger rod 100 as a single molded piece.

With reference to FIGS. 5-7 and with continued reference to FIGS. 2-4, the plunger rod 100 is designed for use as part of a syringe, generally denoted as reference numeral 200. The syringe includes the stopper 118 and the plunger rod 100 positioned within a substantially cylindrical syringe barrel 202. The syringe barrel 202 includes a distal or frontal end 204 which includes an outlet opening 206 and/or a mechanism for attachment of a separate medical device (such as a catheter) and an open proximal or rearward end 208 for receiving the stopper 118 and plunger rod 100 assembly. A retaining structure 210 is provided around the inner diameter of the syringe barrel 202 at the open rearward end 208 thereof. The retaining structure 210 may be configured as a notch or tab extending from the surface of the inner diameter of the syringe barrel 202. However, this is not to be construed as limiting the present invention as the retaining structure 210 may have any suitable shape to engage the retaining anchors 120 of the plunger rod 100.

In operation, as a user pulls the plunger rod 100 out of the open rearward end 208 of the syringe barrel 202, the third portion 126 of the retaining anchor 120 interacts with the retaining structure 210 of the syringe barrel 202 to prevent the plunger rod 100 from being removed from the syringe barrel 202. In addition, this interaction between the retaining anchors 120 and the retaining structure 210 of the syringe barrel 202 prevents the plunger rod 100 from being easily removed from the syringe barrel 202 when the syringe 200 is inadvertently filled up beyond its nominal volume.

The plunger rod 100 may be manufactured from a polymeric material such as, but not limited to, polyolefines (e.g., PE, PP, and their copolymers), polyamides (e.g., nylons), polyesters (e.g., PET), polystyrene, polyurethane, polycarbonate, acrylonitrile-butadiene-styrene, fluoropolymers, ionomers, polyacrylates, or any other similar material. The stopper 118 and the plunger rod 100 may be manufactured integrally or separately and from the same or different material.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of invention which is to be given the full breadth of the claims appended and any and all equivalents thereof.

The invention claimed is:

1. A plunger rod comprising:
an elongated body formed by a plurality of orthogonally spaced elongated ribs extending along a longitudinal axis;
a first ledge adjacent a first end of the elongated body;
a second ledge adjacent a second end of the elongated body, the second end opposing the first end; and
an attachment portion extending from the second ledge,
wherein at least one elongated rib includes a retaining anchor positioned adjacent the second end adjacent the second ledge.

2. The plunger rod of claim 1, wherein each of the elongated ribs include a retaining anchor positioned adjacent the second end adjacent the second ledge.

3. The plunger rod of claim 2, wherein the retaining anchors prevent the elongated body from being removed from a syringe barrel by interacting with a retaining structure provided at an end of the syringe barrel.

4. The plunger rod of claim 1, wherein the retaining anchor includes a first angled portion extending from the second ledge toward the second end of the elongated body, a second portion extending parallel to one of the orthogonally spaced elongated ribs toward the second end of the elongated body, and a third portion extending perpendicularly to one of the orthogonally spaced elongated ribs.

5. The plunger rod of claim 4, wherein the first angled portion extends from the second ledge at an angle of about 3° to about 60°.

6. The plunger rod of claim 4, wherein the third portion of the retaining anchor interacts with a retaining structure of a syringe barrel to prevent the plunger rod from being removed from the syringe barrel.

7. The plunger rod of claim 1, wherein the retaining anchor has a cross-sectional shape selected from the group consisting of rectangular, rounded, or angled shape.

8. The plunger rod of claim 1, wherein the plunger rod comprises a polymeric material selected from the group consisting of: polyolefines, polyamides, polyesters, polystyrene, polyurethane, polycarbonate, acrylonitrile-butadiene-styrene, fluoropolymers, ionomers, polyacrylates, or any combination thereof.

9. The plunger rod of claim 1, wherein the attachment portion is configured to secure a stopper thereto.

10. A syringe comprising:
a substantially cylindrical syringe barrel comprising a fluid dispensing end and an open end, the open end comprising a retaining structure;
a stopper configured to be received within the open end of the syringe barrel; and
a plunger rod configured to engage a portion of the stopper, the plunger rod comprising:
an elongated body formed by a plurality of orthogonally spaced elongated ribs extending along a longitudinal axis;
a first ledge adjacent a first end of the elongated body;
a second ledge adjacent a second end of the elongated body, the second end opposing the first end; and
an attachment portion extending from the second ledge, the attachment portion configured to engage a portion of the stopper,
wherein at least one elongated rib includes a retaining anchor adjacent the second end adjacent the second ledge for engaging the retaining structure of the syringe barrel.

11. The syringe of claim 10, wherein the retaining structure comprises at least one of a notch or tab extending inwardly from an inner surface of the syringe barrel.

12. The syringe of claim 10, wherein each of the elongated ribs include a retaining anchor positioned adjacent the second end adjacent the second ledge.

13. The syringe of claim 10, wherein the at least one retaining anchor includes a first angled portion extending from the second ledge toward the second end of the elongated body, a second portion extending parallel to one of the orthogonally spaced elongated ribs toward the second end of the elongated body, and a third portion extending perpendicularly to one of the orthogonally spaced elongated ribs.

14. The syringe of claim 13, wherein the first angled portion extends from the second ledge at an angle of about 3° to about 60°.

15. The syringe of claim 13, wherein the third portion of the retaining anchor interacts with the retaining structure of the syringe barrel to prevent the plunger rod from being removed from the syringe barrel.

16. The syringe of claim 10, wherein the at least one retaining anchor has a cross-sectional shape selected from the group consisting of: rectangular, rounded, or angled shape.

17. The syringe of claim 10, wherein the plunger rod is manufactured from a polymeric material selected from the group consisting of: polyolefines, polyamides, polyesters, polystyrene, polyurethane, polycarbonate, acrylonitrile-butadiene-styrene, fluoropolymers, ionomers, polyacrylates, or any combination thereof.

18. A plunger rod and stopper assembly adapted for use with a syringe barrel, the assembly comprising:
a stopper; and
a plunger rod configured to engage a portion of the stopper, the plunger rod comprising:
an elongated body formed by a plurality of orthogonally spaced elongated ribs extending along a longitudinal axis;
a first ledge adjacent a first end of the elongated body;
a second ledge adjacent a second end of the elongated body, the second end opposing the first end; and
an attachment portion extending from the second ledge, the attachment portion configured to engage a portion of the stopper,
wherein at least one elongated rib includes a retaining anchor adjacent the second end adjacent the second ledge.

19. The assembly of claim 18, wherein each of the elongated ribs include a retaining anchor positioned adjacent the second end adjacent the second ledge.

20. The assembly of claim 19, wherein the retaining anchors prevent the elongated body from being removed from a syringe barrel by interacting with a retaining structure provided at an end of the syringe barrel.

21. The assembly of claim 18, wherein the at least one retaining anchor includes a first angled portion extending from the second ledge toward the second end of the elongated body, a second portion extending parallel to one of the orthogonally spaced elongated ribs toward the second end of the elongated body, and a third portion extending perpendicularly to one of the orthogonally spaced elongated ribs.

22. The assembly of claim 21, wherein the first angled portion extends from the second ledge at an angle of about 3° to about 60°.

23. The assembly of claim 21, wherein the third portion of the retaining anchor interacts with the retaining structure of the syringe barrel to prevent the plunger rod from being removed from the syringe barrel.

24. The assembly of claim 18, wherein the at least one retaining anchor has a cross-sectional shape selected from the group consisting of: rectangular, rounded, or angled shape.
